(19) United States
(12) Patent Application Publication
(10) Pub. No.: US 2024/0165081 A1
(43) Pub. Date: May 23, 2024

(54) COMPOSITIONS AND METHODS FOR TREATMENT OF INSOMNIA

(71) Applicant: Abraham J. Hoffman, Upper Saddle River, NJ (US)

(72) Inventor: Abraham J. Hoffman, Upper Saddle River, NJ (US)

(21) Appl. No.: 18/514,697

(22) Filed: Nov. 20, 2023

Related U.S. Application Data
(60) Provisional application No. 63/426,997, filed on Nov. 21, 2022.

(51) Int. Cl. A61K 31/4045; A61K 31/015; A61K 31/05; A61K 36/575; A61P 25/00

(52) U.S. Cl. CPC A61K 31/4045; A61K 31/015; A61K 31/05; A61K 36/575; A61P 25/00

(57) ABSTRACT

Compositions for and treatment of insomnia comprising a therapeutically effective amount of an adenosine 2a (A_{2A}) receptor agonist and a therapeutically effective amount of a melatonin receptor agonist. The adenosine 2a receptor agonist is D-limonene and the melatonin receptor agonist is melatonin. Preferably, the composition further comprising Magnolia Bark Extract.

### FIELD OF THE DISCLOSURE

The disclosure relates to the field of insomnia. Particularly, the disclosure relates to compositions for and treatment of insomnia.

### BACKGROUND OF THE INVENTION

Most adults experience regular difficulties with falling and staying asleep, with 80% of US adults reporting that they struggle with sleep at least once a week. Insufficient sleep is well known to have a deleterious impact on physical and mental health, cognition, productivity, and overall quality of life. Consequently, a large market has formed for compositions to alleviate insomnia. About 50% of Americans have reported they have used supplements, medications, or other substances to help them fall asleep.

Dietary supplements are of particular interest to address insomnia. However, while supplements may be a more convenient route for finding a solution for insomnia, supplements generally lack efficacy due to their limited ingredient pool and the complex nature of insomnia. In addition, to be considered a dietary supplement, ingredients must occur within food and/or natural products, limiting options for possible dietary supplement formulations that can adequately address the complexity of insomnia.

There is a need in the art to provide alternative solutions to alleviate insomnia.

### SUMMARY OF THE DISCLOSURE

The present disclosure described herein is an oral sedative comprised of natural products. Advantageously, and in a preferred embodiment, the natural products are dietary supplements. Regulatory authorities generally treat dietary supplements more like food than a pharmaceutical drug. Thus, supplements can be directly marketed and sold over-the-counter to consumers, permitting a rapid move to market, compared to pharmaceutical agents that typically require clinical trials.

Provided herein is a composition comprising a first active component and a therapeutically effective amount of a second active component, the first active component being an adenosine 2a (A_{2A}) receptor agonist and the second active component being a melatonin receptor agonist. The A_{2A} receptor agonist can be D-limonene. A therapeutically effective amount of D-limonene can be from about 100 milligrams (mg) to about 2000 mg.

The melatonin receptor agonist can be melatonin. A therapeutically effective amount of melatonin can be about 0.1 mg to about 20 mg.

The composition can further comprise a therapeutically effective amount of an additional active component chosen from a cannabinoid receptor agonist and/or positive allosteric modulator of GABA-a receptor.

The cannabinoid receptor agonist and/or positive allosteric modulator of GABA-a receptor can be magnolia bark extract, magnolol and/or honokiol. A therapeutically effective amount of honokiol can be about 50 to about 2000 mg. A therapeutically effective amount of magnolia bark extract can be about 50 to about 2000 mg. A therapeutically effective amount of magnolol can be about 50 to about 2000 mg.

The composition can be formulated for oral administration. In some embodiments, at least one of the two active components is formulated as a capsule. In some embodiments, all active components are coformulated together in at least one capsule. In some embodiments, at least two active components are coformulated as separate capsule. In some embodiments, the composition excludes cannabinoids. In some embodiments the composition excludes citrus oil other than orange peel oil.

Provided is a method for treating a sleep disorder in a subject in need thereof, comprising administering to the subject a therapeutically effective amount of an adenosine 2a (A_{2A}) receptor agonist as a first active component and a therapeutically effective amount of a melatonin receptor agonist as a second active component as described above, wherein the therapeutically effective amount is sleep promoting in the subject. In certain embodiments, the A_{2A} receptor agonist and melatonin receptor agonist are administered as a coformulation. In certain embodiments, the coformulation is a composition comprising the A_{2A} receptor agonist and the melatonin receptor agonist in a single vessel, for example a capsule.

The present disclosure also provides an A_{2A} receptor agonist to be administered in combination with a melatonin receptor agonist for use in a combination treatment for treating a sleep disorder. The present disclosure further provides a combination treatment for treating a sleep disorder in a subject in need thereof.

The present disclosure also provides the use of an A_{2A} receptor agonist and a melatonin receptor agonist for treating a sleep disorder. Further provided is the use of an A_{2A} receptor agonist and a melatonin receptor agonist for the preparation of a medicament for treatment of a sleep disorder.

As envisioned in the present disclosure with respect to the disclosed compositions of matter and methods, in one aspect the embodiments of the disclosure comprise the components and/or steps disclosed herein. In another aspect, the embodiments of the disclosure consist essentially of the components and/or steps disclosed herein. In yet another aspect, the embodiments of the disclosure consist of the components and/or steps disclosed herein.

### Definitions

Unless defined otherwise, all technical and scientific terms used herein have the same meaning as commonly understood by one of ordinary skill in the art to which the invention pertains.

Although any methods and materials similar or equivalent to those described herein can be used in the practice of the present disclosure, the preferred materials and methods are described herein. In describing and claiming the present disclosure, the following terminology will be used. It is also to be understood that the terminology used herein is for the purpose of describing particular embodiments and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type.

“About” as used herein when referring to a measurable value such as an amount, a temporal duration, and the like, is meant to encompass variations of +/−20% or +/−10%, more preferably +/−5%, even more preferably +/−1%, and still more preferably +/−0.1% from the specified value, as such variations are appropriate to perform the disclosed methods.

An “effective amount” as used herein, means an amount which provides the indicated therapeutic or prophylactic benefit, i.e., an amount that results in the treatment and/or prevention of insomnia. It is understood, however, that the full therapeutic effect does not necessarily occur by administration of one dose and may occur only after administration of a series of doses. Thus, an effective amount may be administered in one or more administrations. In the context of therapeutic or prophylactic applications, the amount of active agent administered to the subject will depend on the type and severity of the disease or condition and on the characteristics of the subject, such as general health, age, sex, body weight and tolerance to drugs. It will also depend on the degree, severity and type of disease or condition. The skilled artisan will be able to determine appropriate dosages depending on these and other factors. The disclosed compositions can also be administered in combination with one or more additional therapeutic compounds. It will be appreciated by one of skill in the art that various diseases or disorders could require prolonged treatment involving multiple administrations.

As used herein, “individual” or “patient” or “subject” (as in the subject of the treatment) are used interchangeably herein and mean both mammals and non-mammals. Mammals include, for example, humans; non-human primates, e.g. apes and monkeys; dogs; cats; cattle; horses; sheep; and goats. Non-mammals include, for example, fish and birds. The individual is, in certain embodiments, a human being.

As used herein, the term “pharmaceutically acceptable” refers to a formulation of a compound or compounds that does not significantly abrogate the biological activity, a pharmacological activity and/or other properties of the compound or compounds when the formulation of compound or compounds is administered to a patient and is not deleterious to the recipient.

To “treat” a disease as the term is used herein, means to reduce the frequency or severity of at least one sign or symptom of a disease or disorder experienced by a subject. Treating may include one or more of the postponement of further disease progression, reduction in the severity of symptoms that have or are expected to develop, ameliorating existing symptoms, and preventing additional symptoms.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

“Co-administration” as used herein for agents, such as an adenosine 2a (A_{2A}) receptor agonist or melatonin receptor agonist, means that the agents are administered so as to have overlapping therapeutic activities, and not necessarily that the agents are administered simultaneously to the subject. The agents may or may not be in physical combination prior to administration (e.g., in the same capsule). In certain embodiments, the agents are administered to a subject simultaneously or at about the same time. For example, the adenosine 2a (A_{2A}) receptor and the melatonin receptor agonist may be contained in separate capsules, and then ingested simultaneously by the patient, or one capsule at a time, for instance, substantially contiguous in time. As used herein “contiguous in time” means within about within about 5 minutes, within about 4 minutes, within about 3 minutes, within about 2 minutes, within about 1 minute of each other or within 1 minute or less of each other.

“Co-formulated” or “co-formulation” or “coformulation” or “coformulated” as used herein refers to at least two different agents that are formulated together and stored as a combined product in a single vial or vessel (for example a capsule) rather than being formulated and stored individually and then mixed or combined before administration or separately administered. In certain embodiments, the co-formulation contains the adenosine 2a (A_{2A}) receptor and the melatonin receptor agonist. In certain embodiments, the adenosine 2a (A_{2A}) receptor is or comprises D-limonene. In certain embodiments, the melatonin receptor agonist is or comprises melatonin. In certain embodiments, the adenosine 2a (A_{2A}) receptor is D-limonene, and the melatonin receptor agonist is melatonin.

The terms “treatment regimen”, “dosing protocol” and “dosing regimen” are used interchangeably to refer to the dose and timing of administration of each agent in a combination therapy of the disclosure.

As used herein, the term “sleep disorder” refers to problems with the quality, timing and amount of sleep, which cause problems with functioning and distress during the daytime. There is a variety of different types of sleep disorders. Insomnia is the most common type of sleep disorder. Other sleep disorders include narcolepsy, obstructive sleep apnea and restless leg syndrome.

As used herein, the term “insomnia” refers to the difficulty falling asleep and/or difficulty staying asleep, even when a person has the chance to do so. Insomnia is difficulty falling asleep or staying asleep, waking up early, or a disturbance in sleep quality that makes sleep seem inadequate or unrefreshing. Insomnia includes difficulty falling asleep (sleep-onset insomnia) and difficulty staying asleep and waking up earlier than desired (sleep maintenance insomnia). Sleep-onset insomnia includes circadian rhythm sleep disorders, such as delayed sleep phase disorder, shift work disorder, and jet lag. Sleep maintenance insomnia includes sleep apnea or periodic limb movement disorder. The three types of insomnia include transient insomnia (less than one week), acute insomnia (short term), and chronic insomnia (long term).

Primary insomnia is sleeplessness that cannot be attributed to an existing medial, psychiatric or environmental cause, such as drug abuse or medications. Secondary insomnia is when symptoms of insomnia arise from a primary medical illness, mental disorders or other sleep disorders.

As used herein, “promoting sleep” and “sleep promoting activity” means inducing falling asleep (e.g., decreased sleep latency) and/or promoting duration of sleep.

The compositions and methods of the disclosure may be used for the treatment of primary insomnia or secondary insomnia (comorbid with depression or other psychiatric disorders).

“D-limonene” as used herein refers to 4-isopropenyl-1-methylcyclohexene. D-limonene in a major component of citrus oil, and in particular, orange peel citrus oil. In some embodiments herein, the disclosed composition comprising D-limonene excludes non-orange peel citrus oil. D-limonene is commercially available. In some embodiments, the D-limonene is a dietary supplement. In an embodiment, the D-limonene is a food grade D-limonene of about 95% to 97.5% purity.

In preferred embodiments, D-limonene is included as a volatile oil. In preferred embodiments, D-limonene is formulated as a volatile oil and undiluted. In some preferred embodiments, D-limonene is formulated as an undiluted volatile oil within a hermetically sealed oral capsule. In some preferred embodiments, D-limonene is formulated as an undiluted volatile oil and adsorbed to an excipient within an oral capsule. In some preferred embodiments, D-limonene is formulated as an undiluted volatile oil and adsorbed to an excipient within a hermetically sealed oral capsule.

In some embodiments, the composition excludes cannabinoids. As used herein “cannabinoid” refers to the following: THC, CBD, CBC, CBG, CBN, CBL, THCV and CBDV. As used herein, THC″ refers to THCa (tetrahydrocannabiniolic acid) and/or to THC (tetrahydrocannabiniol). As used herein, “CBD” refers to CBDa (cannabidiolic acid) and/or to CBD (cannabidiol). As used herein, “CBG” refers to CBGa (cannabigerolic acid) and/or to CBG (cannabigerol). “As used herein, “CBN” refers to CBNa (Cannabinolic acid) and/or to CBN (cannabinol) unless indicated otherwise. As used herein, “CBC” refers to CBCa (cannabichromenic acid) and/or to CBC (Cannabichromene). As used herein, “CBL” refers to CBLa (Cannabicycol acid) and/or to CBL (Cannabicyclol). As used herein, “THCV” refers to THCVa (tetrahydrocannabivarin acid) and/or to THCV (tetrahydrocannabivarin). As used herein, “CBDV” refers to CBDVa (cannabigerovarin acid) and/or to CBDV (cannabidivarin) unless indicated otherwise.

The term “agonist” as used herein refers to a chemical substance capable of activating a receptor to induce a full or partial pharmacological response. Receptors can be activated or inactivated by either endogenous or exogenous agonists and antagonists, resulting in stimulating or inhibiting a biological response. A physiological agonist is a substance that creates the same bodily responses but does not bind to the same receptor. An endogenous agonist for a particular receptor is a compound naturally produced by the body which binds to and activates that receptor. A superagonist is a compound that is capable of producing a greater maximal response than the endogenous agonist for the target receptor, and thus an efficiency greater than 100%. This does not necessarily mean that it is more potent than the endogenous agonist but is rather a comparison of the maximum possible response that can be produced inside a cell following receptor binding. Full agonists bind and activate a receptor, displaying full efficacy at that receptor. Partial agonists also bind and activate a given receptor but have only partial efficacy at the receptor relative to a full agonist. An inverse agonist is an agent which binds to the same receptor binding-site as an agonist for that receptor and reverses constitutive activity of receptors. Inverse agonists exert the opposite pharmacological effect of a receptor agonist. An irreversible agonist is a type of agonist that binds permanently to a receptor in such a manner that the receptor is permanently activated. It is distinct from a mere agonist in that the association of an agonist to a receptor is reversible, whereas the binding of an irreversible agonist to a receptor is believed to be irreversible. This causes the compound to produce a brief burst of agonist activity, followed by desensitization and internalization of the receptor, which with long-term treatment produces an effect more like an antagonist. A selective agonist is specific for one certain type of receptor.

The term “active” refers to the ingredient, component, or constituent of the compositions of the described invention responsible for the intended therapeutic effect.

The phrase “additional active component” as used herein refers to a component, other than the first and second active components of the described combination treatment that exerts a pharmacological or any other beneficial activity. It is to be understood that “other beneficial activity” can be one that is only perceived as such by the subject using the inventive compositions. Such additional active components include, but are not limited to, a cannabinoid receptor agonist, and a positive allosteric GABA-A receptor modulator.

### DETAILED DESCRIPTION OF THE DISCLOSURE

Embodiments of the present disclosure are described below. It is, however, expressly noted that the present invention is not limited to these embodiments, but rather the intention is that modifications that are apparent to the person skilled in the art and equivalents thereof are also included.

Supplements, such as melatonin capsules, target the circadian rhythm to encourage sleep. However, currently there are no supplements which target the equally crucial phenomenon known as sleep pressure. “Sleep pressure” refers to the brain's steady accumulation of adenosine, a sleep-promoting byproduct of ATP, generated during daytime metabolism. Between the complexity of insomnia, as well as the meager ingredient pool available, insomnia supplements generally lack the efficacy the consumer desires.

The inventor has discovered that a treatment comprising administering a therapeutically effective amount of an adenosine 2a (A_{2A}) receptor agonist as a first active component and a therapeutically effective amount of a melatonin receptor agonist as a second active component is effective in encouraging sleep and targeting sleep pressure. The A_{2A} receptor agonist can be D-limonene. The melatonin receptor agonist can be melatonin. In certain embodiments, the administration is effected by administration of a coformulation of the first active component and the second active component.

The discovery that a composition comprising an adenosine 2a (A_{2A}) receptor agonist such as D-limonene, and a melatonin receptor agonist such a melatonin is efficacious in encouraging sleep and targeting sleep pressure was not expected, for at least the following reasons.

Melatonin is known to encourage sleep. Limonene is thought to have antioxidant, anti-inflammatory, anticancer and anti-heart-disease benefits. See e.g., www.healthline.com/nutrition/d-limonene. However, there is no evidence in the prior art that the oral ingestion of D-limonene has any efficacy on sleep or insomnia. For instance, in a summary of limonene regarding health benefits, side effects uses and dose and precautions, under “side effects” describes only limonene as safe in food amounts and safe in medicinal amounts when taken orally. See e.g., www.rxlist.com/limonene/supplements.htm.

In addition, D-limonene has been investigated as a possible therapeutic agent for treating breast cancer. A study of D-limonene was performed to identify plasma metabolites of limonene (Crowell et al., 1994, Cancer Chemother Pharmacolog 35:31-37). As part of the study, seven healthy patients were administered a supradietary amount of D-limonene (100 mg/kg), which was ingested in custard and blood drawn at the time of ingestion (0 hr), and 4 hr, and 24 hr post ingestion. Mild side effects—eructation and satiety—were noted by several of the seven subjects. One subject noted slight fatigue at the 4 hr time point. The study authors noted that the side effects could be due to the custard used to administer the limonene, rather than to the limonene itself. These results suggest at most that D-limonene may be a short-acting weak sedative even at very large doses (e.g., 100 mg/kg). For instance, a 65 kg person would be administered 6.5 grams of D-limonene.

Limonene is a major aromatic component in citrus-derived essential oils. Citrus-derived essential oils are popular in aromatherapy practices for allegedly providing various calming effects. Limonene has been studied for its potential effects on metabolism and was found to bind to the adenosine A_{2A} receptor (Park et al, 2011, Biochem Biophys Res Commun. 404(1):345-348). A_{2A} receptors are involved in a variety of pathological processes, such a vasodilation, platelet aggregation, reducing inflammation, and sleep process regulation. Park speculated that limonene may bind to A_{2A} receptors in vivo and activate the receptors. However, Park et al. provides no data supporting that limonene affected any of the pathological processes. Moreover, the Crowell reference (Ibid) suggests that while D-limonene may cause fatigue in some persons, it does not have a noticeable sedative effect and there is no suggestion D-limonene can affect sleep.

For at least these reasons, the inventor's discovery that a composition comprising an adenosine₂A (A_{2A}) receptor agonist such as D-limonene, and a melatonin receptor agonist such a melatonin is efficacious in encouraging sleep and targeting sleep pressure was not expected.

The natural agonist of A_{2A} receptors is the sleep-promoting ATP metabolite adenosine, which accumulates in the brain during wakefulness—a phenomenon known as sleep-pressure. These receptors are expressed primarily within the striatum, where upon activation they serve to initiate sleep. Within the striatum, however, A_{2A} receptors fail to sufficiently signal in the absence of melatonin and/or melatonin receptors. Having discovered a composition comprising an adenosine 2A (A_{2A}) receptor agonist such as D-limonene, and a melatonin receptor agonist such a melatonin is efficacious in encouraging sleep and targeting sleep pressure, the inventor believes, without being held to theory, the D-limonene binds to Adenosine 2A (A_{2A}) receptors as an agonist and activates them. By supplying melatonin and D-limonene together as disclosed herein, the inventor believes the agonistic activity of D-limonene is ensured.

### Components and Methods of Use

Provided herein is a composition comprising a first active component and a second active component, the first active component being an adenosine 2A (A_{2A}) receptor agonist and the second active component being a melatonin receptor agonist. In some embodiments, the first active component and the second active component are natural products. In some embodiments, the first active component and the second active are dietary supplements. As shown herein, the composition demonstrates sleep promoting activity.

The A_{2A} receptor agonist can be D-limonene. A therapeutically effective amount of D-limonene can be from about 100 milligrams (mg) to about 2000 mg.

The melatonin receptor agonist can be melatonin. A therapeutically effective amount of melatonin can be about 0.1 mg to about 20 mg.

The composition can further comprise a therapeutically effective amount of an additional active component chosen from one or more of a cannabinoid receptor agonist and/or positive allosteric modulator of GABA-a receptor.

The cannabinoid receptor agonist and/or positive allosteric modulator of GABA-a receptor can be magnolia bark extract, magnolol and/or honokiol. A therapeutically effective amount of honokiol can be about 50 mg to about 2000 mg. A therapeutically effective amount of magnolia bark extract can be about 50 mg to about 2000 mg. A therapeutically effective amount of magnolol can be about 50 mg to about 2000 mg.

Exemplary ranges for exemplary components are shown in Table 1.

TABLE 1

| Example | Range (unit dose) |
|---|---|
| D-limonene (in mg) | about 100 mg to about 2000 mg |
| melatonin (in mg) | 0.3 mg to 20 mg |
| Honokiol (in mg) | about 50 mg to about 2000 mg |
| Magnolia bark extract | about 50 mg to about 2000 mg |
| Magnolol | about 50 mg to about 2000 mg |
| Formulation | One or more capsules; liquid and solid |
| | ingredients separate in multiple capsules; |
| | liquid and solid ingredients combination |
| | filled into one or more capsules, liquid |
| | ingredients adsorbed to a carrier in one or |
| | more capsules; combination of liquid |
| | ingredients in softgels and solid ingredients |
| | in hard capsules |

Exemplary non-limiting examples of the composition disclosed herein are shown in Table 2 as well as Examples 1-19.

TABLE 2

| Example | 1 | 2 | 3 | 4 | 5 | 6 | 7 |
|---|---|---|---|---|---|---|---|
| D-limonene (in mg) | 500 | 500 | 500 | 500 | 500 | 500 | 500 |
| melatonin (in mg) | 7.5 | 7.5 | 5 | 7.5 | 5 | 5 | 5 |
| Honokiol (in mg) | | 600 | | | | | |
| Magnolia bark | | | 500 | | 500 | 300 | 500 |
| extract (in mg) | | | | | | | |
| Magnolol (in mg) | | | | 500 | | | |
| Formulation | Size 00 | D-limonene | D-limonene | D-limonene | Split/ | D-limonene | D-limonene |
| | oral | and melatonin | and melatonin | and melatonin | Co-formulated | was absorbed | was absorbed |
| | capsule | were co- | were co- | were co- | evenly in the | to micro- | to micro- |
| | | formulated | formulated | formulated | two capsules. | crystalline | crystalline |
| | | in one Capsule, | in one capsule, | in one capsule, | D-limonene | cellulose, and | cellulose, and |
| | | Honokiol in a | Magnolia Bark | Magnolol in a | was adsorbed | co-formulated | co-formulated |
| | | separate capsule | Extract in a | separate capsule | to micro- | with melatonin | with melatonin |
| | | | separate capsule | | crystalline | in one capsule, | in one capsule, |
| | | | | | cellulose. | Magnolia Bark | Magnolia Bark |
| | | | | | | Extract in a | Extract in a |
| | | | | | | separate capsule | separate capsule |

The disclosure also provides a method for treating a sleep disorder in a subject in need thereof, comprising administering to the subject a therapeutically effective amount of an adenosine 2a (A_{2A}) receptor agonist as a first active component and a therapeutically effective amount of a melatonin receptor agonist as a second active component as described above, wherein the therapeutically effective amount is sleep promoting in the subject. In some embodiments, the sleep disorder is insomnia. In some embodiments, the sleep disorder is primary insomnia. In some embodiments, the sleep disorder is secondary insomnia.

Treatment efficacy is generally determined by improvement in the symptoms of the sleep disorder. The treatment may be carried out for as long a period as necessary, either in a single, uninterrupted session, or in discrete sessions. The treatment schedule may be repeated as required. According to one embodiment, the composition of the disclosure is administered once daily.

Each therapeutic agent in a combination therapy of the invention may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules.

In some embodiments the coadministration is simultaneous. In some embodiments the coadministration is sequential.

In some embodiments, simultaneous means that the first active component described herein and the second active component described herein are administered to/ingested by the subject less than one hour apart, such as less than 30 minutes apart, less than 15 minutes apart, less than 10 minutes apart or less than 5 minutes apart. In some embodiments, simultaneous means that the first active component described herein and the second active component described herein are administered to the subject at the same time.

In some embodiments, the first active component described herein is administered sequentially with the second active component described herein. In some embodiments, the first active component described herein is administered before administration of the second active component described herein. In some embodiments, the first active component described herein is administered before administration of the second active component described herein. In some embodiments, sequential administration means that the first active component described herein and the second active component described herein are administered at least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart.

A pharmaceutical composition comprises at least one active component and optionally a pharmaceutically acceptable vehicle.

As used herein, the term “pharmaceutically acceptable vehicle” means a chemical composition with which the active component(a) may be combined and which, following the combination, can be used to administer the active component(a) to a subject.

The formulations of the pharmaceutical compositions described herein may be prepared by any method known or hereafter developed in the art of pharmacology. In general, such preparatory methods include the step of bringing the active component(s) into association with a vehicle or one or more additional active components, and/or one or more other accessory ingredients, and then, if necessary or desirable, shaping or packaging the product into a desired single- or multi-dose unit.

Although the descriptions of pharmaceutical compositions provided are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals, including commercially relevant mammals; such as cattle, pigs, horses, sheep, cats, and dogs; birds, including commercially relevant birds such as chickens, ducks, quail, geese, and turkeys; fish including farm-raised fish and aquarium fish; and crustaceans such as farm-raised shellfish and mollusks.

Pharmaceutical compositions that are useful in the methods of the disclosure may be prepared, packaged, or sold in formulations suitable for oral, gastrointestinal, rectal, vaginal, parenteral, topical (including buccal and sublingual), pulmonary, intranasal, buccal, ophthalmic, or another route of administration. Other contemplated formulations include projected nanoparticles, and liposomal preparations. In certain embodiments, the composition is prepared, packaged, or sold in formulations suitable for oral administration. It will be appreciated that the preferred route may vary with for example the condition of the recipient.

A pharmaceutical composition of the invention may be prepared, packaged, or sold in bulk, as a single unit dose, or as a plurality of single unit doses. As used herein, the term “unit dosage” and “unit dosage form” refers to physically discrete units suitable as a unitary dosage for human subjects and other mammals, each unit containing a predetermined quantity of each of the active components calculated to produce the desired therapeutic effect, in association with a suitable pharmaceutical excipient. The amount of each of the active component(s) is generally equal to the dosage of each of the active component(s) which would be administered to a subject or a convenient fraction of such a dosage such as, for example, one-half or one-third of such a dosage. In some embodiments, the unit dosage of each of the active components is divided into multiple capsules, such as two capsules. The unit dosage can be divided equally into two capsules, or can be divided unequally into two capsules.

The relative amounts of the active component(s), the optional pharmaceutically acceptable vehicle, and any additional ingredients in a pharmaceutical composition of the invention will vary, depending upon the identity, size, and condition of the subject treated and further depending upon the route by which the composition is to be administered. By way of example, the composition may comprise between 0.1% and 99.99% (w/w) or between 0.1% and 100% (w/w) active component(s) (e.g., D-limonene and melatonin). A unit dose of a pharmaceutical composition of the invention will generally comprise from about 1 microgram to about 1 g of each active component, and preferably comprises from about 5 micrograms to about 500 milligrams of each active component. The active components may be formulated into dosage forms according to standard practices in the field of pharmaceutical preparations. See Alphonso Gennaro, ed., Remington's Pharmaceutical Sciences, 18th Edition (1990), Mack Publishing Co., Easton, PA.

In addition to the, a pharmaceutical composition of the invention may further comprise one or more additional pharmaceutical agents. Particularly contemplated additional agents include ingredients which can protect the active component(s)s from the effects of the acidic pH environment of portions of the gastrointestinal tract. Substantially all formulations and devices for effecting enteric delivery known or to be developed can be used. Further, as discussed herein, the pharmaceutical composition may contain the one or more optional additional active component(s) (magnolia bark extract, magnolol, honokiol).

Controlled- or sustained-release formulations of a pharmaceutical composition of the invention may be made using conventional technology. In general, a controlled-release preparation is a pharmaceutical composition capable of releasing the active ingredient at the required rate to maintain constant pharmacological activity for a desirable period of time. Such dosage forms provide a supply of an active component to the body during a predetermined period of time and thus maintain drug levels in the therapeutic range for longer periods of time than conventional non-controlled formulations.

A formulation of a pharmaceutical composition of the invention suitable for oral administration may be prepared, packaged, or sold in the form of a discrete solid dose unit including, but not limited to, a tablet (e.g., dragee), a pill, a hard or soft capsule, a cachet, a troche, or a lozenge, each containing a predetermined amount of the active component(s).

Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active component(s) may, for example, be made by compressing or molding the active component(s), optionally with one or more additional ingredients. Compressed tablets may be prepared by compressing, in a suitable device, the active component(s) in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active component(s), a pharmaceutically acceptable vehicle, and at least sufficient liquid to moisten the mixture. Pharmaceutically acceptable excipients used in the manufacture of tablets include, but are not limited to, inert diluents, granulating and disintegrating agents, binding agents, and lubricating agents. Suitable dispersing agents include, but are not limited to, potato starch and sodium starch glycolate. Known surface active agents include, but are not limited to, sodium lauryl sulfate. Known diluents include, but are not limited to, calcium carbonate, sodium carbonate, lactose, microcrystalline cellulose, calcium phosphate, calcium hydrogen phosphate, and sodium phosphate. Suitable granulating and disintegrating agents include, but are not limited to, corn starch and alginic acid. Binding agents include, but are not limited to, gelatin, acacia, pre-gelatinized maize starch, polyvinylpyrrolidone, and hydroxypropyl methylcellulose. Lubricating agents include, but are not limited to, magnesium stearate, stearic acid, silica, and talc.

Tablets may be non-coated or they may be coated using known or to be developed methods to achieve delayed disintegration in the gastrointestinal tract of a subject, thereby providing sustained release and absorption of the active component(s). By way of example, a material such as glyceryl monostearate or glyceryl distearate may be used to coat tablets. Tablets may further comprise a sweetening agent, a flavoring agent, a coloring agent, a preservative, or some combination of these in order to provide pharmaceutically palatable preparation.

Hard capsules comprising the active component(s) may be made using a physiologically degradable composition, such as gelatin. Such hard capsules comprise the active component(s), and may further comprise additional ingredients including, for example, an inert solid diluent such as calcium carbonate, calcium phosphate, or kaolin.

Soft gelatin capsules comprising the active component(s) may be made using a physiologically degradable composition, such as gelatin. Such soft capsules comprise the active component(s), which may be mixed with water or an oil medium such as peanut oil, liquid paraffin, or olive oil.

In some examples, oral compositions may include compounds providing controlled release, sustained release, or prolonged release of the active components. These dosage forms (e.g., tablets or capsules) typically contain conventional and well-known excipients, such as lipophilic, polymeric, cellulosic, insoluble, and/or swellable excipients. Controlled release formulations may also be used for any other delivery sites including intestinal, colon, bioadhesion or sublingual delivery (i.e., dental mucosal delivery) and bronchial delivery. Each of the aforementioned formulations are well known in the art and are described, for example, in the following references: Hansel et al. (1990, Pharmaceutical dosage forms and drug delivery systems, 5th edition, William and Wilkins); Chien 1992, Novel drug delivery system, 2nd edition, M. Dekker); Prescott et al. (1989, Novel drug delivery, J. Wiley & Sons); and Gazzaniga et al., Int. J. Pharm. 1994, 108:77-83.

Oral compositions may be made, using known technology, which specifically release orally-administered agents in the small or large intestines of a human patient. For example, formulations for delivery to the gastrointestinal system, including the colon, include enteric coated systems, based, e.g., on methacrylate copolymers such as poly(methacrylic acid, methyl methacrylate), which are only soluble at pH 6 and above, so that the polymer only begins to dissolve on entry into the small intestine. The site where such polymer formulations disintegrate is dependent on the rate of intestinal transit and the amount of polymer present. For example, a relatively thick polymer coating is used for delivery to the proximal colon (Hardy et al., 1987 Aliment. Pharmacol. Therap. 1:273-280). Polymers capable of providing site-specific colonic delivery can also be used, wherein the polymer relies on the bacterial flora of the large bowel to provide enzymatic degradation of the polymer coat and hence release of the drug. For example, azopolymers (U.S. Pat. No. 4,663,308), glycosides (Friend et al., 1984, J. Med. Chem. 27:261-268) and a variety of naturally available and modified polysaccharides (PCT GB89/00581) can be used in such formulations.

Pulsed release technology such as that described in U.S. Pat. No. 4,777,049 may also be used to administer the active agent to a specific location within the gastrointestinal tract. Such systems permit drug delivery at a predetermined time and can be used to deliver the active agent, optionally together with other additives that may alter the local microenvironment to promote agent stability and uptake, directly to the colon, without relying on external conditions other than the presence of water to provide in vivo release.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active component(s) in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Emulsifying agents include, but are not limited to, lecithin and acacia. Preservatives include, but are not limited to, methyl, ethyl, or n-propyl-para-hydroxybenzoates, ascorbic acid, and sorbic acid. Sweetening agents include, for example, glycerol, propylene glycol, sorbitol, sucrose, and saccharin. Thickening agents for oily suspensions include, for example, beeswax, hard paraffin, and cetyl alcohol.

Liquid solutions of the active component(s) in aqueous or oily solvents may be prepared in substantially the same manner as liquid suspensions, the primary difference being that the active component(s) is dissolved, rather than suspended in the solvent. Liquid solutions of the pharmaceutical composition of the invention may comprise each of the components described with regard to liquid suspensions, it being understood that suspending agents will not necessarily aid dissolution of the active component(s) in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils, such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pennsylvania, U.S.A., which is incorporated herein by reference.

Another aspect of the invention relates to a kit comprising a pharmaceutical composition of the invention and an instructional material. As used herein, an “instructional material” includes a publication, a recording, a diagram, or any other medium of expression which is used to communicate the usefulness of the pharmaceutical composition of the invention for treating, ameliorating, relieving, inhibiting, preventing, or reducing a disorder in a subject or for administering such a composition via a route described herein. The instructional material may also, for example, describe an appropriate dose of the pharmaceutical composition of the invention. The instructional material of the kit of the invention may, for example, be affixed to a container which contains a pharmaceutical composition of the invention or be shipped together with a container which contains the pharmaceutical composition. Alternatively, the instructional material may be shipped separately from the container with the intention that the instructional material and the pharmaceutical composition be used cooperatively by the recipient.

In some embodiments, the pharmaceutical composition is formulated for immediate-release or controlled-release. In some embodiments, the pharmaceutical composition is formulated for delayed-release, extended-release, delayed-extended-release or combinations thereof. In some embodiments, the pharmaceutical composition is formulated in the form of an orally disintegrating tablet.

Extended-release, also known as sustained-release (SR), sustained-action (SA), time-release (TR), controlled-release (CR), modified release (MR), or continuous-release (CR), is a mechanism used in medicine tablets or capsules to dissolve slowly and release the active component(s) over time. The advantages of extended-release tablets or capsules are that they can often be taken less frequently than immediate-release formulations of the same drug, and that they keep steadier levels of the drug in the bloodstream, thus extending the duration of the drug action. Materials and formulation are well known in the art for immediate release, controlled release and extended release compositions. See, for instance USSN 20180264013 (incorporated herein).

The formulation may optionally comprise at least one excipient, such as a filler, a binder, a disintegrant, and/or a flow aid or glidant.

Exemplary fillers include but are not limited to, lactose, glucose, fructose, sucrose, dicalcium phosphate, sugar alcohols also known as “sugar polyol” such as sorbitol, manitol, lactitol, xylitol, isomalt, erythritol, and hydrogenated starch hydrolysates (a blend of several sugar alcohols), corn starch, potato starch, sodium carboxymethycellulose, ethylcellulose and cellulose acetate, enteric polymers, or a mixture thereof.

Exemplary binders, include but are not limited to, water-soluble hydrophilic polymers, such as Povidone (PVP: polyvinyl pyrrolidone), copovidone (a copolymer of polyvinyl pyrrolidone and polyvinyl acetate), low molecular weight HPC (hydroxypropyl cellulose) low molecular weight HPMC (hydroxypropyl methylcellulose), low molecular weight carboxy methyl cellulose, ethylcellulose, gelatin, polyethylene oxide, acacia, dextrin, magnesium aluminum silicate, starch, and polymethacrylates such as Eudragit NE 30D, Eudragit RL, Eudragit RS, Eudragit E, polyvinyl acetate, and enteric polymers, or mixtures thereof.

Exemplary disintegrants include but are not limited to low-substituted carboxymethyl cellulose sodium, crospovidone (cross-linked polyvinyl pyrrolidone), sodium carboxymethyl starch (sodium starch glycolate), cross-linked sodium carboxymethyl cellulose (Croscarmellose), pregelatinized starch (starch 1500), microcrystalline cellulose, water insoluble starch, calcium carboxymethyl cellulose, low substituted hydroxypropyl cellulose, and magnesium or aluminum silicate.

Exemplary glidants include but are not limited to, magnesium, silicon dioxide, talc, starch, titanium dioxide, and the like.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The practice of the invention is illustrated by the following non-limiting examples.

### EXAMPLES

The materials, methods, and embodiments described herein are further defined in the following Examples. Certain embodiments are defined in the Examples herein. It should be understood that these Examples, while indicating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

In the testing of a human subject, melatonin and D-limonene were always mixed in a single capsule. Where multiple capsules were involved, the capsules were applied simultaneously, or immediately in series. For examples involving melatonin, D-limonene, and 300 mg honokiol, the three components were mixed in a single capsule. For examples involving melatonin, D-limonene, and 600 mg honokiol, two capsules were employed; one with mixed melatonin and D-limonene, and the other with 600 mg honokiol. For examples involving melatonin, D-limonene, and 500 mg Magnolol, two capsules were employed; one with mixed melatonin and D-limonene, and the other with 500 mg Magnolol. For examples involving melatonin, D-limonene, and 500 mg Magnolia Bark Extract, two capsules were employed; one with mixed melatonin and D-limonene, and the other with 500 mg Magnolia Bark Extract.

These experiments were conducted under generally similar conditions, including location, time and environment.

In the examples involving testing melatonin, D-limonene, and Magnolia Bark Extract with additional human subjects, microcrystalline cellulose was employed as an inactive carrier to adsorb D-limonene. For one example involving melatonin, D-limonene, and 500 mg Magnolia Bark Extract, all components including microcrystalline cellulose were mixed evenly/coformulated in two capsules. For one example involving melatonin, D-limonene, and 300 mg Magnolia Bark Extract, two capsules were employed; one coformulated with D-limonene, melatonin, and microcrystalline cellulose, and the other with 300 mg Magnolia Bark Extract. For one example involving melatonin, D-limonene, and 500 mg Magnolia Bark Extract, two capsules were employed; one coformulated with D-limonene, melatonin, and microcrystalline cellulose, and the other with 500 mg Magnolia Bark Extract.

### Example 1: Method and Composition-500 mg D-Limonene and 7.5 mg Melatonin

In developing the claimed subject matter, the a combination treatment of 500 mg D-limonene and 7.5 mg melatonin was administered in excess of 100 trials. The following experiences were consistently observed through the trials.

It was noted that ten minutes after ingestion, a feeling of both physical and mental relaxation was observed. The physical and mental relaxation was described as not feeling artificial. It was further noted that what made the relaxation distinct from an organic feeling of relaxation was the state of rest of the subject's muscles and that the muscle relaxation was enjoyable.

Thirty minutes after ingesting the D-limonene and melatonin combination, the above effects were noted to suddenly become much stronger. It was noted that there was a feeling that could be described as hypnosis, as if the brain has fallen asleep before the subject had consciously decided to go to bed. It was also noted that the feeling was the same similar to prior experiences of having slept terribly all week, and then finally fallen into deep sleep one night. This feeling easily lasted for eight to ten hours, and resulted in deep and restorative sleep. Subject also noted that the sleep induced, unlike OTC and prescription sedatives, was characterized by vivid dreams.

Upon waking, the subject noted feeling well rested, awake, and full of energy. The inventor noted that the potency of the D-limonene and melatonin combination appeared comparable to 50 mg diphenhydramine, but notably, the descent into sleep was far smoother, making the combination treatment more effective than an antihistamine. Based upon the subject's experience, it was estimated the D-limonene and melatonin combination treatment was about eighty percent as potent when compared to prescription sedatives such as Zolpidem. Notably, however, unlike GABAergic drugs, the subject never felt “knocked out” but, but rather, just slipped into deep sleep.

Over the numerous trials of the combination treatment of 500 mg D-limonene and 7.5 mg melatonin, the experiences noted were extremely consistent in potency and uniqueness of sensation regarding the psychoactive and sedating nature of the combination.

### Example 2: Method and Composition-500 mg D-Limonene, 7.5 mg Melatonin, and 300 mg-600 mg Honokiol

A variant of the combination treatment: 500 mg D-Limonene, 7.5 mg Melatonin, and 300 mg-600 mg Honokiol was tested in the same human subject as described in Example 1. Honokiol is an example of a cannabinoid receptor agonist and/or positive allosteric modulator of GABA-a receptor.

The subject noted a very pleasant feeling of relaxation prior to falling asleep; however, this combination was less sedating than the embodiment including 1-THP. It is understood that this embodiment could have further recreational or therapeutic value for those who suffer from anxiety. The subject noted that thirty minutes after ingesting this combination, the psychoactive state induced by the D-limonene and melatonin takes effect, with distinct differences believed to be due to the presence of Honokiol. The subject's muscles felt even more relaxed, and his mental state was characterized by a relaxing hypnosis that was much more pronounced than the D-limonene and Melatonin combination alone (Example 1). This feeling was sustained until the subject fell asleep and throughout the night. The subject further noted a slight but discernible feeling of euphoria and clear-headedness.

Based upon the subject's experience, this embodiment appears comparable to a benzodiazepine in terms of its psychoactive effects, but less sedating than drugs such as zolpidem. This embodiment induced better sleep in the subject than OTC antihistamines such as diphenhydramine.

### Example 3: Method and Composition-500 mg D-Limonene, 5 mg Melatonin, and 500 mg Magnolia Bark Extract

An embodiment of the invention containing 500 mg D-limonene, 5 mg melatonin, and 500 mg of Magnolia Bark Extract standardized to 90% neolignans at a 50/50 ratio, thereby providing approximately 225 mg of magnolol and honokiol, was administered to the same subject as Example 1 every night for approximately five months. Magnolol and honokiol are both cannabinoid receptor agonists and positive allosteric modulators of GABA-A receptors, albeit with differing affinities. Magnolol is a stronger cannabinoid than it is a GABA-A positive allosteric modulator. Honokiol is a stronger GABA-A positive allosteric modulator than it is a cannabinoid. The effects of the treatment were consistent, with a slight but insignificant tolerance presenting after three months of daily use.

The subject noted that this embodiment of the treatment was both quite pleasant in terms of anxiolytic properties while sufficiently sedating. The subject noted that thirty minutes after ingesting this combination, the psychoactive state induced by the D-limonene and melatonin takes effect, with distinct differences believed to be due to the presence of Honokiol and Magnolol. This combination was as anxiolytic as the embodiments containing 600 mg Honokiol but was more effectively sedating. The subject also noted that this embodiment was both pleasant to use and effective, as his body was relaxed prior to falling asleep, which occurred approximately one hour following administration. The subject awoke well rested with minimal residual tiredness or brain fog. The efficacy of this embodiment is attributed to the combination of cannabinoid receptor and GABA-A receptor activity induced by the combination of magnolol and honokiol, as both targets potentiate the effects of Adenosine 2a receptor activation.

Based upon the subject's experience, this embodiment is comparable to both a benzodiazepine in terms of its psychoactive effects, and comparable to zolpidem in terms of its sedating effects. This embodiment induced better sleep in the subject than OTC antihistamines such as diphenhydramine.

### Example 4: Method and Composition-500 mg D-Limonene, 7.5 mg Melatonin, and 500 mg Magnolol

An embodiment of the invention containing 500 mg D-limonene, 7.5 mg melatonin, and 500 mg of Magnolol was administered to the same subject as Example 1. Magnolol is a cannabinoid receptor agonist and a positive allosteric modulators of GABA-A receptors. Magnolol is a stronger cannabinoid than it is a GABA-A positive allosteric modulator.

The subject noted that this embodiment of the treatment was both quite pleasant in terms of anxiolytic properties/psychoactive properties. The subject noted that thirty minutes after ingesting this combination, the psychoactive state induced by the D-limonene and melatonin takes effect, with a distinct clear-headedness distinct from all other embodiments/versions of the invention. This combination was found to be as anxiolytic as other embodiments, but surprisingly stimulating in the subject. The subject noted that this embodiment was pleasant to use as his body was relaxed and his mind was clear. Around 60-90 minutes after ingestion, the subject fell asleep. While the subject awoke well rested and experienced minimal residual tiredness or brain fog, the subject noted not feeling as well rested as more sedating embodiments of the invention, including those embodiments including honokiol. The unique qualities of this embodiment are attributed to the cannabinoid stimulating activity of magnolol exceeding GABAergic activity, as activity at cannabinoid type 1 receptors is known to be mildly stimulating.

This embodiment is comparable to a benzodiazepine in terms of its psychoactive effects, and comparable to zolpidem in terms of its sedating effects, based upon the prior experience of the subject. This embodiment induced better sleep in the subject than OTC antihistamines such as diphenhydramine.

### Example 5: Method and Composition-500 mg D-Limonene, 5 mg Melatonin, and 500 mg Magnolia Bark Extract

Subject 2, a 26-year-old male experiencing a bout of severe insomnia, who had previously been prescribed potent sedatives quetiapine and hydroxyzine for sleep, was administered an embodiment of the invention containing 500 mg D-limonene, 5 mg melatonin, and 500 mg magnolia bark extract standardized to 90% neolignans at a 50/50 ratio, thereby providing approximately 225 mg of magnolol and honokiol. The subject was not using any medications or dietary supplements for sleep at this time. Subject 2's experience with this embodiment was consistent with the description provided in Example 3 above. Subject 2 noted the treatment to be quite relaxing and effective for his insomnia. Subject 2 appeared to be slightly less sensitive to the onset of anxiolytic effects generally experienced at the thirty-minute mark following ingestion but was equally receptive to the sedation which presented itself sixty minutes after ingestion. Subject 2 noted this embodiment of the invention was preferable to his prior experience with quetiapine and hydroxyzine, which he had found were less relaxing and had unpleasant side effects. Moreover, Subject 2 felt better rested upon waking with this embodiment than when he had used prescription sedatives previously.

### Example 6: Method and Composition-500 mg D-Limonene, 5 mg Melatonin, and 300 mg Magnolia Bark Extract

Subject 3, a 55-year-old female experiencing secondary insomnia due to tinnitus was administered an embodiment of the invention containing 500 mg D-limonene, 5 mg melatonin, and 300 mg magnolia bark extract standardized to 90% neolignans at a 50/50 ratio, thereby providing approximately 135 mg of magnolol and honokiol. Subject 3 noted minor anxiolytic effects presented themselves at approximately thirty minutes following ingestion. An hour after administration, Subject 3 fell into a deep sleep and slept comfortably for the rest of the night. Upon waking, Subject 3 noted feeling well rested with no residual tiredness. Based upon her prior experience, Subject 3 felt that this embodiment of the invention was far more effective than OTC sleeping medications and supplements such as antihistamines and melatonin for treating her secondary insomnia due to tinnitus

### Example 7: Method and Composition-500 mg D-Limonene, 5 mg Melatonin, and 500 mg Magnolia Bark Extract

Subject 4, a 27-year-old female diagnosed with obsessive compulsive disorder experiencing secondary insomnia due to anxiety was administered an embodiment of the invention containing 500 mg D-limonene, 5 mg melatonin, and 500 mg magnolia bark extract standardized to 90% neolignans at a 50/50 ratio, thereby providing approximately 225 mg of magnolol and honokiol. Subject 4 noted that anxiolytic effects presented themselves at approximately thirty minutes following ingestion, and one hour after administration she fell into a deep sleep. While the embodiment was effective in helping her calm down and fall asleep, Subject 4 noted experiencing especially vivid nightmares. While Subject 4 reported to generally suffer from nightmares, the subject attributed the extra vividness of her nightmares to the enhanced sleep mediated by this embodiment of the invention. The following morning, Subject 4 noted feeling somewhat stressed and not as well rested, which she blamed on her nightmares. Based upon her prior experience, Subject 4 felt that this embodiment of the invention was much more effective for sleep than OTC antihistamines, dietary supplements, and zolpidem. However, Subject 4 noted a reluctancy to use the invention again for sleep due to the especially vivid nightmares she experienced.

### Exemplary Composition 1

A composition comprising 500 mg D-limonene and 7.5 mg melatonin. (See Table 2) The D-limonene and the melatonin can be coformulated. The coformulated composition can be an oral capsule, such as a Size 00 oral capsule.

### Exemplary Composition 2

A composition comprising 500 mg D-limonene, 7.5 mg melatonin, and 600 mg Honokiol. (See Table 2) This composition would be two 00 capsules. One capsule is a coformulation of D-limonene and melatonin. The other capsule contains 600 mg honokiol.

### Exemplary Composition 3

A composition comprising 500 mg D. limonene, 5 mg melatonin and 500 mg Magnolia Bark Extract standardized to 90% neolignans at a 50/50 ratio, thereby providing 225 Magnolol and Honokiol. (See Table 2) One capsule is a coformulation of D-limonene and melatonin. The other capsule contains 600 mg honokiol. The coformulated composition can be an oral capsule, such as a Size 00 oral capsule. The additional oral capsule containing Honokiol is also a Size 00 oral capsule.

### Exemplary Composition 4

A composition comprising 500 mg D-limonene, 7.5 mg melatonin, and 500 mg Magnolol. (See Table 2) One capsule is a coformulation of D-limonene and melatonin. The other capsule contains 500 mg Magnolol. The coformulated composition can be an oral capsule, such as a Size 00 oral capsule. The additional oral capsule containing Magnolol is also a Size 00 oral capsule.

### Exemplary Composition 5

A composition comprising 500 mg D-limonene, 5 mg melatonin and 500 mg Magnolia Bark Extract standardized to 90% neolignans at a 50/50 ratio, thereby providing approximately 225 Magnolol and Honokiol. The composition is evenly split/coformulated in two Size 00 oral capsules. Microcrystaline cellulose is added to each capsule as a carrier/inactive ingredient to adsorb the D-limonene.

### Exemplary Composition 6

A composition comprising 500 mg D-limonene, 5 mg melatonin and 300 mg Magnolia Bark Extract standardized to 90% neolignans at a 50/50 ratio, thereby providing approximately 135 mg Magnolol and Honokiol. One capsule is a coformulation of D-limonene and melatonin. Microcrystaline cellulose is added to this capsule as a carrier/inactive ingredient to adsorb the D-limonene. The other capsule contains 300 mg Magnolia Bark Extract. The coformulated composition can be an oral capsule, such as a Size 00 oral capsule. The additional oral capsule containing Honokiol is also a Size 00 oral capsule.

### Exemplary Composition 7

A composition comprising 500 mg D-limonene, 5 mg melatonin and 500 mg Magnolia Bark Extract standardized to 90% neolignans at a 50/50 ratio, thereby providing approximately 225 Magnolol and Honokiol. One capsule is a coformulation of D-limonene and melatonin. Microcrystaline cellulose is added to this capsule as a carrier/inactive ingredient to adsorb the D-limonene. The other capsule contains 500 mg Magnolia Bark Extract. The coformulated composition can be an oral capsule, such as a Size 00 oral capsule. The additional oral capsule containing Honokiol is also a Size 00 oral capsule.

### Exemplary Composition 8

A composition comprising 400 mg D-limonene and 5 mg melatonin. The D-limonene and the melatonin can be coformulated. The coformulated composition can be an oral capsule, such as a Size 00 oral capsule.

### Exemplary Composition 9

A composition comprising 500 mg D-limonene, 7.5 mg melatonin, and 300 mg Honokiol. The composition is coformulated in one Size 00 oral capsule.

### Exemplary Composition 10

A composition comprising 500 mg D-limonene, 4 mg melatonin and 500 mg Magnolia Bark Extract standardized to 90% neolignans at a 50/50 ratio, thereby providing approximately 225 Magnolol and Honokiol. One capsule is a coformulation of D-limonene and melatonin. Microcrystaline cellulose is added to this capsule as a carrier/inactive ingredient to adsorb the D-limonene. The other capsule contains 500 mg Magnolia Bark Extract. The coformulated composition can be an oral capsule, such as a Size 00 oral capsule. The additional oral capsule containing Honokiol is also a Size 00 oral capsule.

### Exemplary Composition 11

A composition comprising 1000 mg D-limonene, 7.5 mg melatonin and 500 mg Magnolia Bark Extract standardized to 90% neolignans at a 50/50 ratio, thereby providing approximately 225 Magnolol and Honokiol. The D-limonene is contained within two softgels, each softgel containing 500 mg D-limonene, glycerin, purified water, and annato extract as inactive ingredients. Melatonin and Magnolia Bark Extract are coformulated in an additional hard capsule.

### Exemplary Composition 12

A composition comprising 1000 mg D-limonene, 7.5 mg melatonin and 500 mg Magnolia Bark Extract standardized to 90% neolignans at a 50/50 ratio, thereby providing approximately 225 Magnolol and Honokiol. The D-limonene is contained within two softgels, each softgel containing 500 mg D-limonene, glycerin, purified water, and annato extract as inactive ingredients. Melatonin and Magnolia Bark Extract A composition comprising 1000 mg D-limonene, 7.5 mg melatonin and 500 mg Magnolia Bark Extract standardized to 90% neolignans at a 50/50 ratio, thereby providing approximately 225 Magnolol and Honokiol. The D-limonene is contained within two softgels, each softgel containing 500 mg D-limonene, glycerin, purified water, and annato extract as inactive ingredients. Melatonin and Magnolia Bark Extract Extract standardized to 90% neolignans at a 50/50 ratio, thereby providing approximately 225 Magnolol and Honokiol. The D-limonene is contained within two softgels, each softgel containing 500 mg D-limonene, glycerin, purified water, and annato extract as inactive ingredients. Melatonin and Magnolia Bark Extract

### Exemplary Composition 13

A composition comprising 250 mg D-limonene, 10 mg melatonin and 150 mg Magnolia Bark Extract standardized to 90% neolignans at a 50/50 ratio, thereby providing approximately 90 mg Magnolol and 90 mg Honokiol. The composition is coformulated in a single 00 capsule. Microcrystaline cellulose is included in the capsule as a carrier/inactive ingredient to adsorb the D-limonene.

### Exemplary Composition 14

A composition comprised of 500 mg D-limonene, 5 mg melatonin, 600 mg Magnolol, and 600 mg Honokiol. The composition is formulated into three 00 capsules. D-limonene and Melatonin are coformulated in a 00 capsule. Magnolol is contained in an additional 00 capsule. Honokiol is contained in an additional 00 capsule.

### Exemplary Composition 15

A composition comprised of 500 mg D-limonene, 5 mg melatonin, 600 mg Magnolol, and 600 mg Honokiol. The composition is formulated into three capsules. D-limonene and Melatonin are coformulated in a 00 capsule. Magnolol is contained in an additional 00 capsule. Honokiol is contained in an additional 00 capsule.

### Exemplary Composition 16

A composition comprised of 500 mg D-limonene, 0.3 mg melatonin, 250 mg Magnolol, and 250 mg Honokiol. The composition is formulated into three capsules. D-limonene absorbed to microcrystalline cellulose as an inactive carrier in a size 00 capsule. 0.3 mg melatonin is contained in a size 0 capsule. Magnolol and Honokiol are coformulated in a size 00 capsule.

### Exemplary Composition 17

A composition comprised of 250 mg D-limonene, 0.3 mg melatonin, and 250 mg Magnolol. The composition is coformulated within a size 00 capsules. D-limonene adsorbed to microcrystalline cellulose as an inactive carrier.

### Exemplary Composition 18

A composition comprised of 500 mg D-limonene, 20 mg melatonin, 50 mg honokiol, 50 mg magnolol, and 50 mg magnolia bark extract coformulated within a size 00 capsule. D-limonene is adsorbed to microcrystalline cellulose as an inactive carrier.

### Exemplary Composition 19

A composition comprised of 500 mg D-limonene, 5 mg melatonin, and 2000 mg magnolia bark extract is contained within four capsules. D-limonene, melatonin, and 200 mg magnolia bark extract are coformulated in a size 00 capsule. The remaining 1800 mg of magnolia bark extract is divided equally within three size 00 capsules.

### Exemplary Composition 20

A composition comprised of 500 mg D-limonene, 5 mg melatonin, and 2000 mg Honokiol is contained within four capsules. D-limonene, melatonin, and 200 mg Honokiol are coformulated in a size 00 capsule. The remaining 1800 mg of Honokiol is divided equally within three size 00 capsules.

### Exemplary Composition 21

A composition comprised of 500 mg D-limonene, 5 mg melatonin, and 2000 mg Magnolol is contained within four capsules. D-limonene, melatonin, and 200 mg Magnolol are coformulated in a size 00 capsule. The remaining 1800 mg of Magnolol is divided equally within three size 00 capsules.

### Exemplary Composition 22

A composition comprised of 100 mg D-limonene, 0.3 mg melatonin, 100 mg Magnolol, 100 mg Honokiol, and 150 mg of Magnolia Bark Extract are coformulated within a size 00 capsule.

While the novel technology has been illustrated and described in detail in the foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, and accounts, this discussion should by no means be interpreted as limiting the technology. All patents, patent applications, and references to texts, scientific treatises, publications, and the like referenced in this application are incorporated herein by reference in their entirety.

### REFERENCES

Crowell, P. L., Lin, S., Vedejs, E., & Gould, M. N. (1992). Identification of metabolites of the antitumor agent d-limonene capable of inhibiting protein isoprenylation and cell growth. Cancer Chemotherapy and Pharmacology, 31(3), 205-212.
Igimi, H., Nishimura, M., Kodama, R., & Ide, H. (1974). Studies on the metabolism of d-limonene (p-mentha-1,8-diene). I. The absorption, distribution and excretion of d-limonene in rats. Xenobiotica; the Fate of Foreign Compounds in Biological Systems, 4(2), 77-84. https://doi.org/10.3109/00498257409049347
Kodama, R., Yano, T., Furukawa, K., Noda, K., & Ide, H. (1976). Studies on the metabolism of d-limonene (p-mentha-1,8-diene). IV. Isolation and characterization of new metabolites and species differences in metabolism. Xenobiotica; the Fate of Foreign Compounds in Biological Systems, 6(6), 377-389. https://doi.org/10.3109/00498257609151649
Park, H. M., Lee, J. H., Yaoyao, J., Jun, H. J., & Lee, S. J. (2011). Limonene, a natural cyclic terpene, is an agonistic ligand for adenosine A(2A) receptors. Biochemical and Biophysical Research Communications, 404(1), 345-348. https://doi.org/10.1016/j.bbrc.2010.11.121
Satoh, S., Matsumura, H., Koike, N., Tokunaga, Y., Maeda, T., & Hayaishi, O. (1999). Region-dependent difference in the sleep-promoting potency of an adenosine A_{2A} receptor agonist. The European Journal of Neuroscience, 11(5), 1587-1597.
Scammell, T. E., Gerashchenko, D. Y., Mochizuki, T., McCarthy, M. T., Estabrooke, I. V., Sears, C. A., Saper, C. B., Urade, Y., & Hayaishi, O. (2001). An adenosine A2a agonist increases sleep and induces Fos in ventrolateral preoptic neurons. Neuroscience, 107(4), 653-663.
Song, Y., Seo, S., Lamichhane, S., Seo, J., Hong, J. T., Cha, H. J., & Yun, J. (2021). Limonene has anti-anxiety activity via adenosine A2A receptor-mediated regulation of dopaminergic and GABAergic neuronal function in the striatum. Phytomedicine: International Journal of Phytotherapy and Phytopharmacology, 83, 153474. https://doi.org/10.1016/j.phymed.2021.153474
Vigushin, D. M., Poon, G. K., Boddy, A., English, J., Halbert, G. W., Pagonis, C., Jarman, M., & Coombes, R. C. (1998). Phase I and pharmacokinetic study of D-limonene in patients with advanced cancer. Cancer Research Campaign Phase I/II Clinical Trials Committee. Cancer Chemotherapy and Pharmacology, 42(2), 111-117.
Sun_2007_Limonene-Safety and Clinical Applications, Alternative Medicine Review Vo. 12, No. 3
Limonene_Uses, Benefits, Side Effects, and Dosage_www.healthline.com
Limonene www.rxlist.com/limonene/supplements.htm #SideEffects (downloaded Jan. 20, 2022)

What is claimed is:

1. A composition comprising a therapeutically effective amount of a first active component and a therapeutically effective amount of a second active component, the first active component being an adenosine 2a (A_{2A}) receptor agonist and the second active component being a melatonin receptor agonist.

2. The composition of claim 1, wherein the A_{2A} receptor agonist is D-limonene.

3. The composition of claim 2, wherein the therapeutically effective amount of D-limonene is from about 100 milligram (mg) to about 2000 mg.

4. The composition of claim 1, wherein the melatonin receptor agonist is melatonin.

5. The composition of claim 4, wherein the therapeutically effective amount of melatonin is about 0.3 mg to about 20 mg.

6. The composition of claim 1, further comprising a therapeutically effective amount of an additional active component chosen from one or more of a cannabinoid receptor agonist and positive allosteric modulator of GABA-a receptor.

7. The composition of claim 6, where the cannabinoid receptor agonist or positive allosteric modulator of GABA-a receptor is magnolia bark extract, magnolol or honokiol.

8. The composition of claim 7, wherein the therapeutically effective amount of honokiol is about 50 to about 2000 mg.

9. The composition of claim 7, wherein the therapeutically effective amount of magnolia bark extract is about 50 to about 2000 mg.

10. The composition of claim 7, wherein the therapeutically effective amount of magnolol is about 50 to about 2000 mg.

11. The composition of claim 2 wherein the D-limonene is a volatile oil.

12. The composition of claim 1, formulated for oral administration.

13. The composition of claim 12, wherein at least one active component is formulated as a capsule.

14. The composition of claim 13, wherein all active components are formulated together as a capsule.

15. The composition of claim 13, wherein at least two active components are formulated as separate capsules.

16. The composition of claim 12, formulated as an aqueous or oily suspension, an aqueous or oily solution, or an emulsion.

17. The composition of claim 1, wherein the composition excludes cannabinoids and non-orange peel citrus oil.

18. The composition of claim 1, consisting of a therapeutically effective amount of an adenosine 2a (A_{2A}) receptor agonist, and a therapeutically effective amount of a melatonin receptor agonist, optionally an additional active component chosen from one or more of a dopamine antagonist, a cannabinoid receptor agonist and/or positive allosteric modulator of GABA-a receptor, and a cannabinoid Type 2 receptor activator, and optionally one of more non-active components selected from one or more of: a pharmaceutically acceptable carrier, an aqueous or oily solvent, a dispersing or wetting agent, a suspending agent, a preservative and an excipient.

19. The composition of claim 1, selected from:
(a) 500 mg of D-limonene volatile oil and 7.5 mg of melatonin, optionally contained within a capsule;
(b) 500 mg of D-limonene volatile oil, 5 mg of melatonin, and 500 mg of Magnolia Bark Extract, divided into two capsules
(c) 500 mg of D-limonene volatile oil, 5 mg of melatonin, and 300 mg of Magnolia Bark Extract, optionally contained within a capsule or divided into two capsules;

20. A method for treating a sleep disorder in a subject in need thereof, comprising administering to the subject a composition of claim 1, wherein the therapeutically effective amount is sleep promoting in the subject.

21. A method for treating a sleep disorder in a subject in need thereof, comprising administering to the subject a combination treatment comprising a therapeutically effective amount of an adenosine 2a (A_{2A}) receptor agonist and a therapeutically effective amount of a melatonin receptor agonist, wherein the therapeutically effective amount is sleep promoting in the subject.

22. The method of claim 21, wherein the A_{2A} receptor agonist is D-limonene.

23. The method of claim 22, wherein the therapeutically effective amount of D-limonene is from about 100 milligram (mg) to about 2000 mg.

24. The method of claim 21, wherein the melatonin receptor agonist is melatonin.

25. The method of claim 24, wherein the therapeutically effective amount of melatonin is about 0.1 mg to about 20 mg.

26. The method of claim 21, wherein the A_{2A} receptor agonist and melatonin receptor agonist are administered in the same composition.

27. The method of claim 21, wherein the combination treatment is a multi-component treatment further comprising a therapeutically effective amount of an additional active component chosen from a cannabinoid receptor agonist and/or positive allosteric modulator of GABA-a receptor.

28. The method of claim 27, where the cannabinoid receptor agonist and/or positive allosteric modulator of GABA-a receptor is magnolia bark extract, magnolol and/or honokiol.

29. The method of claim 28, wherein the therapeutically effective amount of honokiol is about 50 to about 2000 mg.

30. The method of claim 28, wherein the therapeutically effective amount of magnolia bark extract is about 50 to about 2000 mg.

31. The method of claim 28, wherein the therapeutically effective amount of magnolol is about 50 to about 2000 mg.

32. The method of claim 21, wherein the administering is by oral administration.

33. The method of claim 21, wherein the sleep disorder is insomnia.
